(19) United States
(12) Patent Application Publication
(10) Pub. No.: US 2005/0008604 A1
(43) Pub. Date: Jan. 13, 2005

(54) Modified soy proteins in personal care compositions

(76) Inventors: Thomas M. Schultz, Randolph, NJ (US); Huu Tam Tran, St. Louis, MO (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 10/868,332

(22) Filed: Jun. 15, 2004

Related U.S. Application Data
(60) Provisional application No. 60/479,330, filed on Jun. 17, 2003.

(51) Int. Cl. A61K 7/06; A61K 7/11; A61K 35/78; A61K 7/42

(52) U.S. Cl. 424/70.14; 424/757; 424/59

(57) ABSTRACT

Personal care compositions comprising a modified soy protein as an active ingredient are described. The modified soy proteins of the instant invention include soy proteins modified by treatment with a reducing agent and subsequently reacted with a carboxylic acid anhydride; soy proteins modified by treatment with a reducing agent, reacted with a carboxylic acid anhydride and subsequently oxidized; soy proteins modified by reaction with a hydroxy alkyl acrylate; soy proteins modified with ionic monomers such as epoxide, acrylate, and chlorohydrin; and soy proteins modified by reaction with an alkyl acrylimidoglycolate. The compositions may be in the form of skin care, skin cleansing, or anti-wrinkle products, shampoos, make-up, conditioners, lotions, aerosols, gels, mousses, dyes, or bleaches.

### CROSS-REFERENCE TO RELATED APPLICATION

This application claims the benefit of U.S. Provisional Application Ser. No. 60/479,330, filed on Jun. 17, 2003, which is hereby incorporated by reference in its entirety.

### FIELD OF THE INVENTION

The invention relates to the field of personal care products. More specifically, the invention relates to the use of modified soy proteins in compositions for skin care, hair care, hair coloring, and other personal care products.

### BACKGROUND OF THE INVENTION

Film-forming substances are widely used in compositions for skin care and hair care as conditioning agents and moisturizers, and to protect the skin and hair against environmental and chemical damage. These substances adsorb onto and/or absorb into the skin or hair, forming a protective coating. Commonly used film-forming substances include synthetic polymers, such as silicones, polyvinylpyrrolidone, acrylic acid polymers, and polysaccharides, and proteins, such as collagen, keratin, elastin, silk, and soy proteins. Many proteins are known to be particularly effective film-forming agents, depending on their molecular weight. Because of their low solubility at the conditions used in skin and hair care products, proteins are commonly used in the form of peptides, formed by the hydrolysis of the protein.

In hair care compositions, film-forming substances are used to form a protective film on the surface of the hair to protect it from damage due to grooming and styling, shampooing, and exposure to ultraviolet light and the reactive chemicals commonly used in permanent wave agents, hair coloring products, bleaches, and hair straighteners, which denature the hair keratin protein. Moreover, these film-forming substances improve the elasticity of the hair. Film-forming substances that have been used in hair care products include proteins, such as keratin, collagen, and silk proteins and hydrolysates thereof, and polymeric materials, such as polyacrylates, long chain alkyl quaternized amines, and siloxane polymers. For example, Cannell at al. in U.S. Pat. No. 6,013,250 describe a hair care composition for treating hair against chemical and ultraviolet light damage. This composition comprises hydrolyzed protein, having an abundance of sulfur containing amino acids, and divalent cations. Omura et al. in U.S. Pat. No. 6,139,851 describe a hair care cosmetic for treating split ends which contains one or more types of silicone derivatives, one or more types of specific polyether modified silicone, and a lower alcohol. The major problem with these compositions is that they lack the required durability required for long-lasting protection.

Film-forming substances are also used in skin care compositions to form a protective film on the skin. These films can serve to lubricate and coat the skin to passively impede the evaporation of moisture and smooth and soften the skin. Commonly used film-forming substances in skin care compositions include hydrolyzed animal and vegetable proteins (Puchalski et al., U.S. Pat. No. 4,416,873, El-Menshawy et al., U.S. Pat. No. 4,482,537, and Kojima et al., JP 02311412) and silk proteins (Philippe et al., U.S. Pat. No. 6,280,747 and Fahnestock et al., copending U.S. Provisional Patent Application No. 60/448,952).

The use of hydrolyzed soy proteins as film-forming agents in personal care compositions is well known (See for example Russ et al. U.S. Pat. No. 6,299,890, Burns et al. U.S. Pat. No. 5,635,168, Simon et al. U.S. Pat. No. 6,284,233, and Yoshida et al. U.S. Pat. No. 6,036,730). In most of these compositions, the soy proteins are used in an extensively hydrolyzed form due to the low solubility of the intact proteins. The use of some types of chemically modified soy proteins in personal care compositions is also known. For example, Jones et al. in U.S. Pat. No. 5,412,074 describe the use of silicone-modified soy proteins and peptides, formed by reaction with an organofunctional silicone reagent, as a film-forming agent in cosmetic formulations. Hale et al. in GB 1,529,841 describe the use of hydrolyzed soy proteins, which are acylated by reaction with an acid anhydride, in detergent compositions. Marsh et al. in CA 1051312 describe soy protein hydrolyzates that are modified by treatment with an oxidizing agent such as hydrogen peroxide and their use in cosmetic or detergent compositions. In that disclosure, the purpose of the peroxide treatment is to bleach the material to a pale color, thereby making it more cosmetically acceptable. Isao et al. in published application JP 2001031697A describe soy protein hydrolyzates that are modified by reaction with an alkyl or alkenyl glycidyl ether and their use in detergent compositions. Additionally, Alain et al. in FR 2707647 describe modified soy proteins formed by coupling an α-hydroxy acid with a soy protein or peptide and their use in cosmetic and pharmaceutical compositions.

Although the soy proteins known in the art provide a conditioning effect in hair and skin care compositions, there is a need for improved film-forming agents that provide superior durability and long-lasting protection from the various activities mentioned above that cause hair damage. Additionally, there is a need for improved film-forming agents for use in skin care compositions that provide a superior smooth and durable, protective coating as well as added strength to the skin.

Applicants have met the stated need by unexpectedly discovering that certain modified soy proteins, originally developed for industrial applications, form deposits onto natural hair keratin while retaining moisture to provide a conditioning feel along with flexibility. Applicants have also discovered that these modified soy proteins form deposits onto the skin while retaining moisture to provide a conditioning feel along with a toning or skin tightening effect. Additionally, the water solubility of these modified soy proteins facilitates their incorporation into hair care, skin care and other personal care compositions.

### SUMMARY OF THE INVENTION

The invention provides for a personal care composition comprising:
an effective amount of a soy protein selected from the group consisting of soy proteins modified by treatment with a reducing agent and subsequently reacted with a carboxylic acid anhydride; soy proteins modified by treatment with a reducing agent, reacted with a carboxylic acid anhydride and subsequently oxidized; soy proteins modified by reaction with a hydroxy alkyl acrylate; soy proteins modified with epoxide, acrylate, or chlorohydrin ionic monomers; soy proteins modified by reaction with an alkyl acrylimidoglycolate; and mixtures thereof.

The composition may also contain at least one cosmetic adjuvant selected from the group consisting of fillers, surfactants, thixotropic agents, antioxidants, preserving agents, dyes, pigments, fragrances, thickeners, vitamins, hormones, moisturizers, UV absorbing organic sunscreens, UV scattering inorganic sunscreens, wetting agents, cationic polymers, anionic polymers, nonionic polymers, amphoteric polymers, and hair coloring active substances.

The effective amount of the modified soy protein is preferably from about 0.001 to about 90% by weight of the total weight of the composition, about 0.001 to about 60% by weight of the total weight of the composition, or about 0.005 to about 20% by weight of the total weight of the composition.

The personal care composition described above can be an anhydrous composition containing a fatty phase, the fatty phase containing at least one liquid, solid, or semi-solid fatty substance, or in the form of an aqueous solution containing mono or polyhydric alcohols. If the composition is an anhydrous composition, the fatty substance preferably is selected from the group consisting of isododecane, hydrogenated polyisobutene, squalane, isononyl isononanoate, cyclotetra- and -pentadimethicones, phenyltrimethicone, ethylene homopolymers, ethoxylated fats and oils, fluoroalkanes, microcrystalline waxes, ozocerite, beeswax, seracite, shea butter, candelilla wax, arachidyl propionate, fluoropolymers represented by the monomer X₁X₂C═CX₃F, wherein X₁, X₂, and X₃ are independently H or F, and copolymers of ethylene and of at least one monomer represented by the formula CH₂═CH—R₃, wherein R₃ is an alkyl radical containing from 1 to 30 carbon atoms or an aryl or aralkyl radical.

Alternatively, the personal care composition described above can be in the form of a creme emulsion, a gel, a dry powder, an aerosol, a mousse, an alcohol-in-oil emulsion, an alcohol and water solution, an aqueous solution, or an emulsion solution. Preferably, these compositions further comprise a carbomer, gum, or other thickener in a proportion of from about 0.1 to about 15% by weight relative to the total weight of the composition. Alternatively, at least one of the materials found in the composition is in the form of a powder and the effective amount of the modified soy protein is present along with a pigment or filler. More preferably, the effective amount of the modified soy protein is from about 0.001 to about 60% by weight of the total weight of the composition.

Additionally, the hair coloring composition described above can be in the form of an aqueous alkaline solution suitable for preparing compositions for coloring or bleaching hair. Preferably, the hair coloring composition described above further comprises a direct hair dye, an oxidative hair dye, or a peroxygen-based oxidizing agent for bleaching hair.

The personal care composition described above can also be in the form of a stable dispersion of water-in-oil or oil-in-water type.

Additionally, the invention provides a method for forming a protective film of soy protein on skin, hair, lips or nails comprising:
(a) applying to the skin, hair, lips, or nails the personal care composition described above; and
(b) allowing the formation of the soy protein protective film on the skin, hair, lips or nails.

The invention also provides a method for coloring or bleaching hair comprising applying the aqueous alkaline solution described above to the hair for a period of time sufficient to cause the coloration of the hair. Preferably, the aqueous alkaline solution is applied to the hair for a period of about 5 to about 50 minutes.

### DETAILED DESCRIPTION OF THE INVENTION

The present invention comprises personal care compositions comprising an effective amount of a modified soy protein in a suitable cosmetic carrier. The personal care compositions of the present invention include any composition that may be applied to the skin, hair, lips, or nails to provide a cosmetic or beneficial effect. These personal care compositions include, but are not limited to, skin care, skin cleansing, make-up, facial lotions; creme moisturizers; body washes; body lotions; foot and hand cremes; lipsticks; eyeshadow; foundations; facial powders; deodorants; shaving creme compositions, including gel types; nail polish; shaving lotions; creme and lotion depilatories; facial masks made with clay materials; and anti-aging products; and hair care compositions such as shampoos, hair conditioners, hair treatment creams, styling gels and foams, hair mousses, hair sprays, set lotions, blow-styling lotions, hair color lotions, hair relaxing compositions, permanent wave first agents and permanent wave second agents.

The following definitions are used herein and should be referred to for interpretation of the claims and the specification.

The terms “polypeptide” and “protein” are used interchangeably.

The term “peptide” is used to describe a subunit of a polypeptide or protein formed by hydrolysis.

The term “substantially unhydrolyzed soy proteins” is used to refer to soy proteins that have no significant reduction in the individual subunit molecular weight of the protein material.

The term “partially hydrolyzed soy protein” is used to describe soy proteins that are hydrolyzed to a limited extent to form peptides with a molecular weight in the range of about 2 kDa to about 350 kDa.

The term “natural protein” is used to describe a protein that is isolated from its native animal or plant source.

The term “recombinant protein” is used to describe a protein that is produced by a non-native organism using genetic engineering techniques.

Both natural proteins and recombinant proteins can exist in their native sequences or in sequences modified by genetic engineering.

Soy protein materials that are useful for preparing the modified soy proteins of the instant invention include soy flour, soy concentrate and most preferably soy protein isolate and native isolated soy protein. The soy flour, soy concentrate, soy protein isolate, and native isolated soy protein are formed from a soybean starting material, which may be soybeans or a soybean derivative. The soybean starting material may be soybean cake, soybean chips, soybean meal or preferably, soybean flakes. These soybean starting materials may be prepared using conventional procedures known in the art. The soy protein isolate is preferably prepared from defatted soy flake material, as described by Krinski et al. in U.S. Pat. No. 5,766,331 (in particular, column 4, lines 41 to 60), which is incorporated herein by reference. The soy protein material is then chemically modified to form the modified soy proteins of the instant invention.

The modified soy proteins of the instant invention include, but are not limited to soy proteins modified by treatment with a reducing agent and subsequently reacted with a carboxylic acid anhydride; soy proteins modified by treatment with a reducing agent, reacted with a carboxylic acid anhydride and subsequently oxidized; soy proteins modified by reaction with a hydroxy alkyl acrylate; soy proteins modified with ionic monomers such as epoxide, acrylate, and chlorohydrin; and soy proteins modified by reaction with an alkyl acrylimidoglycolate. The soy proteins of the instant invention may be used in a substantially unhydrolyzed condition or may be partially hydrolyzed. As used herein, a substantially unhydrolyzed soy protein refers to a soy protein that has no significant reduction in the individual subunit molecular weight of the protein material. Substantially unhydrolyzed soy proteins have a molecular weight from about 10 kDa to about 600 kDa. It is preferred that a substantially unhydrolyzed soy protein be used in the personal care compositions of the instant invention. The larger protein molecules make better surface-modifying agents than the extensively hydrolyzed soy proteins described in the prior art. However, if desired, partially hydrolyzed soy proteins may be used. Methods useful for the partial hydrolysis of soy proteins are well known in the art (see for example Yoshioka et al., U.S. Pat. No. 5,753,214), and include, but are not limited, to treatment with acid or base, or with a proteolytic enzyme such as pepsin, papain, trypsin, chymotrypsin, subtilisin, and the like. The time of reaction and the concentration of the reagents may be adjusted to obtain the degree of hydrolysis desired. The partially hydrolyzed soy proteins of the instant invention have a molecular weight of about 2 kDa to about 350 kDa.

The soy proteins modified by treatment with a reducing agent and subsequently reacted with a carboxylic acid anhydride may be prepared using the method described by Coco et al. in U.S. Pat. No. 4,474,694 (in particular, column 3, line 57 to column 6, line 15), which is incorporated herein by reference. In that method, an isolated soy protein material is initially prepared by treatment of defatted soybean flakes with an alkaline solution to solubilize the protein. This alkaline treatment step produces three important modifications in the proteins, including 1) hydrolysis of the amides of asparagine and glutamine to carboxylic acids, 2) protein cross-linking through beta elimination of serine and cysteine, followed by reaction with lysine, and 3) protein reorganization by hydrophilic and hydrophobic groups. The protein cross-linking results in the formation of some very high molecular weight fragments, such that the molecular weight range of the modified proteins is from about 2 kDa to about 5,000 kDa. The protein extract is then separated from the alkali-insoluble solids by filtration or centrifugation. This protein extract or dispersion is used as the starting material in the process. This protein extract or dispersion is first treated with a reducing agent such as thioglycolic acid or salts of thioglycolic acid to react with the disulfide bonds and improve the reactivity of the protein material during subsequent reaction. This treated soy protein dispersion may be kept in substantially unhydrolyzed form or may be partially hydrolyzed as described supra. The treated soy protein dispersion is then reacted with a carboxylic acid anhydride to modify the protein material and impart unique film-forming properties for the protein material which are useful in personal care compositions, and makeup.

The preferred carboxylic acid anhydride is a dicarboxylic acid anhydride such as phthalic anhydride or succinic anhydride. Following the reaction of the soy protein material with the carboxylic acid anhydride, the modified soy protein may be isolated by acid precipitation at the isoelectric point and recovered. These types of modified soy proteins are commercially available, for example from E.I. du Pont de Nemours and Co., Inc. (Wilmington, Del.).

The soy proteins modified by treatment with a reducing agent, reacted with a carboxylic acid anhydride and subsequently oxidized may be prepared by the method described by Krinski et al. in U.S. Pat. No. 4,961,788 (in particular, column 2, line 58 to column 3, line 12), which is incorporated herein by reference. In that method, soy proteins modified by treatment with a reducing agent and subsequently reacted with a carboxylic acid anhydride are prepared according the method described by Coco et al, supra. This modified soy protein is then further modified by oxidation using a bleaching agent, such as hydrogen peroxide. These types of modified soy proteins are commercially available, for example from E.I. du Pont de Nemours and Co., Inc. (Wilmington, Del.).

The soy proteins modified by reaction with a hydroxy alkyl acrylate may be prepared by the method described by Steinmetz et al. in U.S. Pat. No. 4,687,826 (in particular, column 3, line 8 to column 4, line 41), which is incorporated herein by reference. In that method, an isolated soy protein material is initially prepared by treatment of defatted soybean flakes with an alkaline solution to solubilize the protein. The protein extract is then separated from the alkali-insoluble solids by filtration or centrifugation. The alkaline dispersion of the soy protein may be maintained in substantially unhydrolyzed form or may be partially hydrolyzed, as described supra. The soy protein dispersion is then reacted with a hydroxyl alkyl acrylate in an amount sufficient to modify the protein. The preferred hydoxy alkyl acrylate is one in which the alkyl group is C₁ to C₈, and most preferably, C₂ or C₃. The modified soy protein may be isolated by acid precipitation at the isoelectric point and recovered.

The soy proteins modified with ionic monomers such as epoxide, acrylate, and chlorohydrin may be prepared using the method described by Krinski et al. in U.S. Pat. No. 4,689,381 (in particular, column 3, line 9 to column 4, line 53), which is incorporated herein by reference. In that method, an isolated soy protein material is initially prepared by treatment of defatted soybean flakes with an alkaline solution to solubilize the protein. The protein extract is then separated from the alkali-insoluble solids by filtration or centrifugation. The alkaline dispersion of the soy protein may be maintained in substantially unhydrolyzed form or may be partially hydrolyzed, as described supra. The soy protein dispersion is then reacted with a cationic epoxide monomer, such as 2,3 epoxypropyltrimethyl ammonium chloride, a cationic acrylic monomer, such as methacrylamidopropyltrimethyl ammonium chloride, or a cationic chlorohydrin monomer, such as 3-chloro, 2-hydroxypropyltrimethyl ammonium chloride, in an amount sufficient to modify the protein material. The modified soy protein may be isolated by acid precipitation at the isoelectric point and recovered. These types of modified soy proteins are commercially available, for example from E.I. du Pont de Nemours and Co., Inc. (Wilmington, Del.).

The soy proteins modified by reaction with an alkyl acrylimidoglycolate may be prepared by the method described by Krinski et al. in U.S. Pat. No. 4,554,337 (in particular, column 3, line 7 to column 4, line 31), which is incorporated herein by reference. In that method, an isolated soy protein material is initially prepared by treatment of defatted soybean flakes with an alkaline solution to solubilize the protein. The protein extract is then separated from the alkali-insoluble solids by filtration or centrifugation. The soy protein dispersion is then reacted with an alkyl acrylamidoglycolate alkyl ether, such as methyl acrylamidoglycolate methyl ether, in an amount sufficient to modify the protein material. The modified soy protein may be isolated by acid precipitation at the isoelectric point and recovered.

In the compositions of the present invention, the expression “effective amount” of a modified soy protein corresponds to a proportion of from about 0.001 to about 90% by weight, but preferably from about 0.001 to about 60% by weight, most preferably from about 0.005 to about 20% by weight, relative to the total weight of the composition. This proportion may vary as a function of the type of personal care composition.

The personal care compositions of the present invention also contain one or more conventional cosmetic or dermatological additives or adjuvants, including, but not limited to, fillers, surfactants, thixotropic agents, antioxidants, preserving agents, dyes, pigments, fragrances, thickeners, vitamins, hormones, moisturizers, UV absorbing organic sunscreens, UV scattering inorganic sunscreens, wetting agents, cationic, anionic, nonionic or amphoteric polymers, and hair coloring active substances. These adjuvants are well known in the field of cosmetics and are described in many publications, for example see Harry's Book of Cosmeticology, 8^{th} edition, Martin Rieger, ed., Chemical Publishing, New York (2000).

Among these adjuvants, the fillers are generally present in personal care products in a maximum proportion of about 99.9% by weight relative to the total weight of the composition. These fillers, in the form of very fine powders, can be of natural or synthetic origin and include, but are not limited to, mineral powders, such as talc, kaolin, mica, silica, silicates, alumina, zeolites, hydroxyapatite, sericite, titanium dioxide, titanium micas, barium sulfate, calcium carbonate, calcium sulfate, bismuth oxychloride, boron nitride and metal powders such as aluminum powder; plant powder, such as corn starch, wheat starch or rice starch powders; organic powders, such as polyamide powder, polyester powder, polytetrafluoroethylene powder, the powder of fluorinated alkanes, polyethylene powder and other inert plastics. These various powders can also be coated, for example with metal salts of fatty acids, amino acids, lecithin, collagen, silicone compounds, fluoro compounds or with any common coating agent.

The personal care compositions of this invention may also contain surfactants or wetting agents, preferably at about 0.001 to about 18%, more preferably at about 0.005 to about 15% by weight of the total composition. The terms “surfactants” and “wetting agents” as used herein refer to surface-active agents which, when added to water, cause it to penetrate more easily into, or spread on the surface of another material, by reducing the surface tension of the water at the water-air or water-oil interface. By “surface active agent” is meant any compound that reduces surface tension when dissolved in water or water solutions. The selection of a surfactant for this purpose presents a wide range of possibilities known in the art. Suitable surfactants include, but are not limited to, the following:
(1) anionic surfactants, such as metallic or alkanolamine salts of fatty acids for example sodium laurate and triethanolamine oleate; alkyl benzene sulfones, for example triethanolamine dodecyl benzene sulfonate; alkyl sulfates, for example sodium lauryl sulfate; alkyl ether sulfates, for example sodium lauryl ether sulfate (2 to 8 EO); sulfosuccinates, for example sodium dioctyl sulfonsuccinate; monoglyceride sulfates, for example sodium glyceryl monostearate monosulfate; isothionates, for example sodium isothionate; methyl taurides, for example Igepon T; acylsarcosinates, for example sodium myristyl sarcosinate; acyl peptides, for example Maypons and lamepons; acyl lactylates, polyalkoxylated ether glycollates, for example trideceth-7 carboxylic acid; phosphates, for example sodium dilauryl phosphate.
(2) cationic surfactants, such as amine salts, for example sapamin hydrochloride; quatenary ammonium salts, for example Quaternium 5, Quaternium 31 and Quaternium 18;
(3) amphoteric surfactants, such as imidazol compounds, for example Miranol; N-alkyl amino acids, such as sodium cocaminopropionate and asparagine derivatives; betaines, for example cocamidopropylebetaine
(4) nonionic surfactants, such as fatty acid alkanolamides, for example oleic ethanolamide; esters or polyalcohols, for example Span; polyglycerol esters, for example that esterified with C.sub.1 2.sub.-1 8 fatty acids and one or several OH groups; Polyalkoxylated derivatives, for example polyoxy:polyoxyethylene stearate (available for example from McIntyre Co); ethers, for example polyoxyethe lauryl ether (available for example from Stepan Co., Northfield, Ill., as Stepanol ES); ester ethers, for example Tween; amine oxides, for example coconut and dodecyl dimethyl amine oxides. Mixtures of two or more of the above surfactants can be employed in the compositions according to the invention.

The personal care compositions of this invention may also contain thixotropic or gelling agents, preferably at about 0.02 to about 20%, more preferably at about 0.05 to about 18% by weight of the total composition. Suitable thixotropic or gelling agents include, but are not limited to, stearates of aluminum, calcium, magnesium, potassium, sodium, or zinc; hydroxystearate, isostearate, laurate, linoleate, myristate, oleate, olivate, palmate, palmitate, tallowate, rosinate, and the like, and fatty acid esters of glycol, triglycerides, mixtures of fatty alcohols, cholesterol derivatives and in particular hydroxycholesterol, and clay minerals which swell in the presence of oil, and in particular those belonging to the montmorillonite group.

The personal care compositions of this invention may also contain antioxidants, preferably at about 0.001 to about 10%, more preferably at about 0.01 to about 8% by weight of the total composition. Suitable antioxidants are ingredients which assist in preventing or retarding spoilage. Examples of antioxidants suitable for use in the compositions of the invention include, but are not limited to, potassium sulfite, sodium bisulfite, sodium erythrobate, sodium metabisulfite, sodium sulfite, propyl gallate, cysteine hydrochloride, butylated hydroxytoluene, butylated hydroxyanisole, and the like.

The personal care compositions of this invention may also contain preserving agents, preferably at about 0.001 to about 8%, more preferably at about 0.01 to about 5% by weight of the total composition. Suitable preserving agents include, but are not limited to, benzoic acid, benzyl alcohol, benzylhemiformal, benzylparaben, 5-bromo-5-nitro-1,3-dioxane, 2-bromo-2-nitropropane-1,3-diol, butyl paraben, phenoxyethanol, methyl paraben, ethyl paraben, propyl paraben, diazolidinyl urea, calcium benzoate, calcium propionate, captan, chlorhexidine diacetate, chlorhexidine digluconate, chlorhexidine dihydrochloride, chloroacetamide, chlorobutanol, p-chloro-m-cresol, chlorophene, chlorothymol, chloroxylenol, m-cresol, o-cresol, DEDM Hydantoin, DEDM Hydantoin dilaurate, dehydroacetic acid, diazolidinyl urea, dibromopropamidine diisethionate, DMDM Hydantoin, Phenonip®, Kathon® and all of those disclosed on pages 570 to 571 of the CTFA Cosmetic Ingredient Handbook, Second Edition, 1992, which is herein incorporated by reference.

The personal care compositions of this invention may also contain dyes, preferably at about 0.1 to about 15%, by weight of the total composition. Suitable dyes include, but are not limited to, eosin derivatives such as D&C Red No. 21 and halogenated fluorescein derivatives such as D&C Red No. 27, D&C Red Orange No. 5 in combination with D&C Red No. 21 and D&C Orange No. 10. The personal care compositions of this invention may also contain pigments, preferably at about 0.1 to about 15% by weight of the total composition. Suitable pigments may be inorganic or organic or alternatively metal lakes and include, but are not limited to, titanium dioxide, zinc oxide, barium oxide, D&C Red No. 36 and D&C Orange No. 17, the calcium lakes of D&C Red Nos. 7, 11, 31 and 34, the barium lake of D&C Red No. 12, the strontium lake D&C Red No. 13, the aluminum lakes of FD&C Yellow No. 5, of FD&C Yellow No. 6, of D&C Red No. 27, of D&C Red No. 21, and of FD&C Blue No. 1, iron oxides, manganese violet, chromium oxide, ultramarine blue, and carbon black particles.

The personal care compositions of this invention may also contain fragrances, preferably at about 0.01 to about 10%, by weight of the total composition. Numerous fragrances, both natural and synthetic, are well known in the art. For example, Secondini (Handbook of Perfumes and Flavors, Chemical Publishing Co., Inc., New York, 1990), incorporated herein by reference, describes many of the natural and synthetic fragrances used in cosmetics. Suitable natural fragrances include, but are not limited, to jasmines, narcissus, rose, violet, lavender, mint, spice, vanilla, anise, amber, orange, pine, lemon, wintergreen, rosemary, basil, and spruce. Suitable synthetic fragrances include, but are no limited to, acetaldehyde, C7 to C16 alcohols, benzyl acetate, butyric acid, citric acid, isobutyl phenyl acetate, linalyl butyrate, malic acid, menthol, phenyl ethyl cinnamate, phenyl propyl formate, tannic acid, terpineol, vanillin, amyl salicylate, benzaldehyde, diphenyl ketone, indole, and the like.

The personal care compositions of this invention may also contain thickeners, preferably at about 0.001 to about 25%, more preferably at about 0.1 to about 15%, by weight of the total composition. Suitable thickeners include, but are not limited to, starch; gums, such as gum arabic or xanthan gum; carbomer polymers, such as Carbopol® 941, 940, 934 (available from Union Carbide Co., Midland, Mich.), and Ultrez 10; kaolin or other clays, ethylene glycol monostearate, carboxyvinyl polymer, acrylic copolymers, hydroxyethyl cellulose, and hydroxypropyl cellulose. The personal care compositions of this invention may also contain vitamins and/or coenzymes, preferably at about 0.001 to about 10%, more preferably at about 0.01% to about 8%, most preferably at about 0.05% to about 5% by weight of the total composition. Suitable vitamins include, but are not limited to, ascorbic acid and derivatives thereof; the B vitamins, such as thiamine, riboflavin, pyridoxin, and the like; vitamin A and derivatives thereof; vitamin E and derivatives thereof; vitamin D and vitamin K; as well as coenzymes such as thiamine pyrophosphate, flavin adenine dinucleotide, folic acid, pyridoxal phosphate, tetrahydrofolic acid, and the like.

The personal care compositions of this invention may also contain hormones, preferably at about 0.0001 to about 0.01% by weight of the total composition. Suitable hormones include, but are not limited to, estrogen, progesterone, pregnenolone, testosterone, estradiol, hydrocortisone, and cortisone.

The personal care compositions of this invention may also contain moisturizers, preferably at about 0.1 to about 30%, more preferably at about 0.5 to about 25%, most preferably at about 1 to about 20% by weight of the total composition. These moisturizers include water-soluble, low molecular weight moisturizers, fat-soluble, low molecular weight moisturizers, water-soluble, high molecular weight moisturizers and fat-soluble, high molecular weight moisturizers. Suitable water-soluble, low molecular weight moisturizers include, but are not limited to, serine, glutamine, sorbitol, mannitol, pyrrolidone-sodium carboxylate, glycerin, propylene glycol, 1,3-butylene glycol, ethylene glycol, polyethylene glycol (polymerization degree n=2 or more), polypropylene glycol (polymerization degree n=2 or more), polyglycerin (polymerization degree n=2 or more), lactic acid and lactate. Suitable fat-soluble, low molecular weight moisturizers include, but are not limited to, cholesterol and cholesterol ester. Suitable water-soluble, high molecular weight moisturizers include, but are not limited to, carboxyvinyl polymers, polyaspartate, tragacanth, xanthane gum, methyl cellulose, hydroxymethyl cellulose, hydroxyethyl cellulose, hydroxypropyl cellulose, carboxymethyl cellulose, water-soluble chitin, chitosan and dextrin. Suitable fat-soluble, high molecular weight moisturizers include, but are not limited to, polyvinylpyrrolidone-eicosene copolymers, polyvinylpyrrolidone-hexadecene copolymers, nitrocellulose, dextrin fatty acid ester and high molecular silicone.

The personal care compositions of this invention may also contain UV absorbing organic sunscreens, preferably at about 0.001 to about 20%, more preferably at about 0.01 to about 10%, most preferably at about 0.05 to about 8% by weight of the total composition. UV absorbing organic sunscreens are herein defined as organic chemicals that absorb ultraviolet light of wavelengths between 290 and 329 nm. Suitable UV absorbing organic sunscreens include, but are not limited to, para-aminobenzoic acid, ethyl para-aminobenzoate, amyl para-aminobenzoate, octyl para-aminobenzoate, ethylene glycol salicylate, phenyl salicylate, octyl salicylate, benzyl salicylate, butylphenyl salicylate, homomenthyl salicylate, benzyl cinnamate, 2-ethoxyethyl para-methoxycinnamate (such as Parsol® available from Givaudan-Roure Co.), octyl para-methoxycinnamate, glyceryl mono(2-ethylhexanoate) dipara-methoxycinnamate, isopropyl para-methoxycinnamate, diisopropyl-diisopropylcinnamic acid ester mixtures, urocanic acid, ethyl urocanate, hydroxymethoxybenzophenone, hydroxymethoxybenzophenonesulfonic acid and salts thereof, dihydroxymethoxybenzophenone, sodium dihydroxymethoxybenzophenonedisulfonate, dihydroxybenzophenone, tetrahydroxybenzophenone, 4-tert-butyl-4′-methoxydibenzoylmethane, 2,4,6-trianilino-p-(carbo-2′-ethylhexyl-1′-oxy)-1,3,5-triazine, and 2-(2-hydroxy-5-methylphenyl)benzotriazole. UV scattering inorganic sunscreen materials, such as inorganic pigments and metal oxides, including but not limited to oxides of titanium (such as SunSmart available from Cognis Corp), zinc, and iron, may also be incorporated into the compositions of the instant invention. UV scattering inorganic sunscreens are herein defined as inorganic substances that scatter ultraviolet light of wavelengths between 210 and 280 nm. These UV scattering inorganic sunscreens may be used in the personal care compositions of this invention at concentrations of preferably about 0.001 to about 40%, more preferably at about 0.01 to about 10%, most preferably at about 0.05 to about 8% by weight of the total composition.

The personal care compositions of this invention may also contain other film-forming polymers, preferably at about 0.01 to about 20%, more preferably at about 0.01% to about 10%, by weight of the total composition. These polymers serve as conditioners to coat the skin or hair, or to coat particles that are present in the composition. These polymers may be cationic, anionic, nonionic, or amphoteric. Cationic polymers are herein defined as synthetic or natural polymers that contain, or have been modified to contain, positively charged groups and/or groups that can ionize to positively charged groups. Suitable cationic polymers, include, but are not limited to, cationized cellulose, cationized guar gum, diallyly quaternary ammonium salt/acrylamide copolymers, quaternized polyvinylpyrrolidone and derivatives thereof, polyquaternium-1, polyquaternium-2, polyquaternium-5, polyquaternium-6, polyquaternium-7, polyquaternium-8, polyquaternium-9, polyquaternium-11, polyquaternium-12, polyquaternium-13, polyquaternium-14, polyquaternium-115, polyquaternium-16, polyquaternium-17, polyquaternium-18, polyquaternium-19, polyquaternium-20, polyquaternium-22, polyquaternium-27, polyquaternium-28, polyquaternium-29, polyquaternium-30, and mixtures thereof, wherein the compound designation is the name adopted for the compound by the Cosmetic, Toiletry and Fragrance Association, and found in the CTFA International Cosmetic Ingredient Dictionary, J. Nikitakis, ed., Cosmetic, Toiletry and Fragrance Association, Inc., Washington, D.C. (1991).

Anionic polymers are herein defined as synthetic or natural polymers that contain, or have been modified to contain, negatively charged groups and/or groups that can ionize to negatively charged groups. Suitable anionic polymers, include, but are not limited to, polyacrylic acid, polymethacrylic acid, carboxymethylcellulose, hydroxymethylcellulose, and starch.

Nonionic polymers are herein defined as synthetic or natural polymers that do not contain any charged groups. Suitable nonionic polymers, include, but are not limited to, polyvinylpyrrolidone, polyvinyl alcohol, polyethylene glycol, polyvinylacetate, polysiloxanes, and copolymers of vinylpyrrolidone and vinyl acetate.

Amphoteric polymers are herein defined as synthetic or natural polymers that contain both negatively and positively charged groups and/or groups that can ionize to give positively and negatively charged groups. Suitable amphoteric polymers are described by Marchi et al. In U.S. Pat. No. 5,643,672, incorporated herein by reference. Examples include, but are not limited to, polymers resulting from the copolymerization of a monomer derived from a vinyl compound carrying a carboxyl group, such as acrylic acid, methacrylic acid, maleic acid and alpha-chloroacrylic acid, and a basic monomer derived from a substituted vinyl compound containing at least one basic nitrogen atom, such as dialkylaminoalkyl methacrylates and acrylates and dialkylaminoalkylmethacrylamides and-acrylamides, products sold by the company National Starch under the name Amphomer®,methyl methacrylate/ethyldimethylcarboxymethylammonium methacrylate copolymers, such as the products sold by Chimex under the name Mexomer PX (CTFA name: “polyquaternium-30”), methacryloylethylbetaine/methacrylate copolymer sold by Sandoz under the name Diaformer, the methacryloylethylbetaine/methacrylate copolymer sold by Amerchol under the name Amersette, polysiloxane polyorganobetaine copolymers sold by Goldschmidt under the name Abil B 9950 (CTFA Name: “Dimethicone PropylPG-Betaine”), the polydimethylsiloxane containing alkylphosphobetaine groups sold by Siltech under the name Pecosil SPB-1240, and the oxyethyleneoxypropylene organobetaine/siloxane copolymer sold by Goldschmidt under the name BC 1610.

The personal care compositions of the present invention may also include an effective amount of one or more other natural or recombinant proteins that enhance the film-forming and coating properties of the modified soy proteins or provide other desirable properties. An effective amount of these additional proteins is herein defined as a proportion from about 0.001 to about 60% by weight, preferably from about 0.01 to about 20% by weight relative to the total weight of the composition. This proportion may vary as a function of the type of personal care composition. Every protein molecule may be considered to be a polymer of amino acids. There are 20 naturally occurring amino acids, each with a common backbone combined with one of 20 variable side chains. Amino acids are commonly classified as either neutral, anionic or cationic amino acids based on the charge of the variable side chain. The naturally occurring anionic amino acids include aspartic acid and glutamic acid, both of which have a carboxylate group at the termini of their respective variable side chains. The naturally occurring cationic amino acids include lysine, arginine and histidine, all of which comprise an amino group in their respective variable side chains. Amino acids may also be modified to result in a net cationic charge. All such amino acids are considered herein to be “cationic amino acids”.

The most advantageous proteins to use in the composition will depend on the specific composition. For example, skin care and hair care products are generally in the pH range of 2.0 to 10.0. Proteins having a predominance of anionic amino acids, relative to cationic amino acids, are likely to be negatively charged at the general pH range of these products. Conversely, proteins having a relative abundance of cationic amino acids are likely to be positively charged under similar conditions. It is preferred that the amino acid composition of the additional natural or recombinant proteins of the present invention contain a preponderance of anionic amino acids relative to cationic amino acids, so that the proteins are negatively charged at the general pH range of skin care and hair care products. Specifically, the compositions of the present invention preferably comprise an additional natural or recombinant protein, wherein the molar ratio of anionic amino acids to cationic amino acids is at least 1.1:1, preferably from about 1.1:1 to 25:1, and more preferably from about 1.1:1 to 15:1. The term “molar ratio of anionic amino acids to cationic amino acids” of the natural or recombinant protein in the present invention is herein defined as the total molar amount of anionic amino acids (for example, adding the individual molar amount of the anionic amino acids, such as aspartic acid and glutamic acid) in relation to the total molar amount of cationic amino acids (for example, adding the individual molar amount of lysine, arginine and histidine) in the protein. The molar ratio of anionic amino acids to cationic amino acids may be readily calculated based on the overall amino acid composition of the natural or recombinant protein or mixtures of natural or recombinant proteins. Such compositions are frequently known in the art. Alternatively, the amino acid composition of any protein may be determined using techniques known to those skilled in the art, which include, but are not limited to automated amino acid analysis and high pressure liquid chromatography. Such techniques separate the individual amino acids, and the molar ratio of anionic to cationic amino acids may be determined.

Furthermore, the additional natural or recombinant proteins for use in the compositions of the present invention may comprise sulfur-containing amino acids to provide a protective effect against ultraviolet light or chemical damage to the skin and hair. The preferred natural or recombinant proteins of this invention are characterized in that they contain at least about 0.25%, preferably from about 0.25 to about 15%, and more preferably from about 0.25 to about 5.0% by weight of a sulfur containing amino acid. The term “sulfur-containing amino acids” is herein defined as any amino acid, natural or synthetic, containing sulfur in any form, including, but not limited to, sulfhydryl groups or disulfide bonds. Sulfur-containing amino acids suitable for the present invention include, but are not limited to, cysteine, cystine, methionine, and their respective derivatives and synthetic analogues.

The preferred natural or recombinant proteins for use in this invention include, but are not limited to, wheat and oat proteins, collagen, keratins, gelatin, elastin, fibronectin, soluble reticulin, and water-soluble silk proteins, prepared as described by Fahnestock in copending U.S. Provisional Patent Application No. 60/425,617. The natural or recombinant proteins may be used in their native form or they may be modified to alter their ionic charge, for example by sulfonation or succinylation. These proteins may also be in partially hydrolyzed form by cleaving them into smaller peptide units, as described supra. These additional proteins may also include unmodified soy proteins in unhydrolyzed or hydrolyzed form.

According to one embodiment of the present invention, the compositions are anhydrous and comprise a fatty phase in a proportion generally of from about 10 to about 90% by weight relative to the total weight of the composition, wherein the fatty phase contains at least one liquid, solid or semi-solid fatty substance. The fatty substances include, but are not limited to oils, fats, waxes, gums, and so-called pasty fatty substances. The oils in the fatty phase may be of mineral, animal, plant or synthetic origin, and may or may not be volatile at room temperature. Oils of mineral origin include, but are not limited to, liquid paraffin and liquid petroleum jelly. Oils of animal origin include, but are not limited to, squalene and squalane. Oils of plant origin include, but are not limited to, sweet almond oil, beauty-leaf oil, palm oil, avocado oil, jojoba oil, sesame oil, olive oil, castor oil and cereal germ oils such as, for example, wheatgerm oil. Synthetic oils include, but are not limited to:
(1) esters of the following formula:
   R₁—COOR₂
   in which:
R₁ represents a higher fatty acid residue containing from 7 to 20 carbon atoms, and
R₂ represents a hydrocarbon-based radical containing from 3 to 30 carbon atoms.
   These esters, include, but are not limited to: purcellin oil, butyl myristate, isopropyl myristate, cetyl myristate, isopropyl palmitate, butyl stearate, hexadecyl stearate, isopropyl stearate, octyl stearate, isocetyl stearate, decyl oleate, hexyl laurate, isononyl isononanoate and esters derived from lanolic acid, such as isopropyl lanolate and isocetyl lanolate. Other synthetic oils include, but are not limited to, isododecane (available for example from Exxon-Mobil Chemical Co., Houston, Tex., under the trade name of ISOPAR™, isohexadecane, polyisobutenes and hydrogenated polyisobutene, as well as acetylglycerides, octanoates and decanoates of polyalcohols such as those of glycol and of glycerol, ricinoleates of alcohols or of polyalcohols, such as cetyl ricinoleate, propylene glycol dicaprylate and diisopropyl adipate;
(2) fatty alcohols including, but not limited to, oleyl alcohol, linoleyl alcohol, linolenyl alcohol, isostearyl alcohol and octyldodecanol;
(3) ethoxylated oils and fats, including but not limited to, triglycerides with a polyethylene glycol chain inserted, ethoxylated mono and di-glycerides, polyethoxylated lanolins, ethoxylated butter derivatives, polyethylene glycol derivatives of glyceryl cocoate, glyceryl caproate, glyceryl caprylate, glyceryl tallowate, glyceryl palmate, glyceryl stearate, glyceryl laurate, glyceryl oleate, glyceryl ricinoleate, and glyceryl fatty esters derived from triglycerides, such as palm oil, almond oil, and corn oil, glyceryl tallowate, glyceryl cocoate, and polyethylene glycol based polyethoxylated fatty alcohols such as PEG 40 hydrogenated castor oil (commercially available under the tradename Cremophor (RTM) from BASF), PEG 7 glyceryl cocoate and PEG 20 glyceryl laurate (commercially available from Henkel under the tradenames Cetiol (RTM) HE and Lamacit (RTM) GML 20 respectively), and polyethylene glycol ethers of ceteryl alcohol such as Ceteareth 25 (available from BASF under the trade name Cremaphor A25).
(4) silicone oils including, but not limited to, optionally functionalized linear polydiorganosiloxanes, cyclic polydiorganosiloxanes and in particular cyclotetra- and -pentadimethicones and organopolysiloxanes such as alkyl, alkoxy or phenyl dimethicones, and in particular phenyltrimethicone (available from Dow Corning, Midland, Mich., as Simethicone and DC 200 Fluids);
(5) fluoro oils including, but not limited to, fluoroalkanes and fluoropolyethers, partially fluorinated hydrocarbon-based oils, and fluoropolymers represented by the monomer unit:
   X₁ X₂C═CX₃F
   wherein X₁, X₂, and X₃ are independently H or F.

The waxes in the fatty phase may be of mineral, fossil, animal, plant or synthetic origin or alternatively can be hydrogenated oils or fatty esters which are solid at 25° C. The mineral waxes, include, but are not limited to, microcrystalline waxes, paraffin, petroleum jelly and ceresine. The fossil waxes, include, but are not limited to, ozocerite and montan wax. The waxes of animal origin, include, but are not limited to beeswax, spermaceti, lanolin wax and derivatives obtained from lanolin such as lanolin alcohols, hydrogenated lanolin, hydroxylated lanolin, acetylated lanolin, lanolin fatty acids and acetylated lanolin alcohol. The waxes of plant origin, include, but are not limited to, candelilla wax, carnauba wax, Japan wax and cocoa butter. The synthetic waxes, include, but are not limited to, ethylene homopolymers, seracite, shea butter, and copolymers of ethylene and of a monomer corresponding to the formula:
CH₂═CH—R₃
in which: R₃ represents an alkyl radical containing from 1 to 30 carbon atoms or an aryl or aralkyl radical. The alkyl radical of 1 to 30 carbon atoms is preferably a methyl, ethyl, propyl, isopropyl, butyl, decyl, dodecyl or octadecyl radical. Waxes obtained by Fisher-Tropsch synthesis and silicone waxes may also be used.

The hydrogenated oils which are solid at 25° C. include, but are not limited to, hydrogenated castor oil, hydrogenated palm oil, hydrogenated tallow and hydrogenated coconut oil. The fatty esters which are solid at 25° C. include, but are not limited to, propylene glycol monomyristate and myristyl myristate. Waxes which can be used in the compositions according to the invention include, but are not limited to, cetyl alcohol, stearyl alcohol, mono-, di- and triglycerides which are solid at 25° C., stearic monoethanolamide, colophony and its derivatives such as glycol abietate and glyceryl abietate, sucroglycerides and calcium, magnesium, zinc and aluminum oleates, myristates, lanolates, stearates and dihydroxystearates.

The pasty-type fatty substances can be of mineral, animal, plant or synthetic origin. The pasty fatty substances include, but are not limited to, synthetic esters such as arachidyl propionate, polyvinyl laurate, polyethylene waxes and organopolysiloxanes such as alkyldimethicones, alkoxydimethicones or dimethicone esters.

These anhydrous compositions can be in various forms including, but not limited to, an oily gel, solid products, such as compacted or cast powders, or alternatively sticks such as, for example lipsticks. When the compositions according to the present invention are in the form of an oily gel, they generally contain a thixotropic or gelling agent, examples of which are given supra. The thixotropic agents can be present in various proportions depending on the desired texture of the compositions. However, in most cases, they are present in a proportion of from about 1 to about 20% by weight relative to the total weight of the composition.

The anhydrous compositions of the present invention may be used in particular as skin care, skin cleansing, or make-up products. When they are present in the form of make-up products, they can be foundations, mascaras, eyeliners, lipsticks, eyeshadows or blushers. These compositions are generally colored and contain dyes and/or pigments as cosmetic adjuvants, which are described supra.

According to a another embodiment of the present invention, the compositions are stable dispersions in the form of a water-in-oil (W/O) or oil-in-water (O/W) emulsion, which comprise: a fatty phase, as described supra, in a proportion of from about 0.1 to about 50% by weight relative to the total weight of the emulsion, the said fatty phase containing at least one modified soy protein, in a proportion of from about 0.001 to about 60% by weight relative to the total weight of the emulsion; an aqueous phase in a proportion of from about 50 to about 98.9% by weight relative to the total weight of the emulsion; and at least one emulsifier in a proportion of from about 1 to about 10% by weight relative to the total weight of the emulsion. Suitable emulsifiers are well known in the field of cosmetic products. For example, water-in-oil emulsifiers include, but are not limited to, sterols such as cholesterol and its associated esters and alcohols, lanolin, calcium oleate and other fatty acid soaps of divalent metals, beeswax, and polyhydric alcoholics of fatty acids such as glyceryl monostearate and sorbitan sesquioleate. Suitable oil-in-water emulsifiers include, but are not limited to, ordinary soaps, partially sulfated fatty alcohols, Cetomacrogol B.P., polyethoxylated esters known as Spans, cetydimethylbenzyl ammonium chloride, and gums and gum substitutes

These emulsions, which are in the form of creams, have good film-forming properties and give a very satisfactory sensation after they have been applied. Such emulsions can be used as skin care, skin cleansing, or make-up products. When these compositions are skin care products, they can be anti-wrinkle products for improving the appearance of the skin. When these compositions are make-up products, they may be foundations or mascaras, containing a certain proportion of the pigments and/or dyes described supra.

In another embodiment of the present invention, the compositions are hair care compositions. Hair care compositions are herein defined as compositions for the treatment of hair, including but not limited to shampoos, conditioners, hair treatment creams, aerosols, gels, hair sprays, set lotions, blow styling lotions, hair relaxing compositions, permanent wave first agents, permanent wave second agents, and mousses. The hair care compositions of the present invention comprise an effective amount of modified soy protein in a cosmetically acceptable medium. An effective amount of a modified soy protein for use in a hair care composition is herein defined as a proportion of from about 0.001 to about 90% by weight relative to the total weight of the composition. Components of a cosmetically acceptable medium for hair care compositions are described by Omura et al. in U.S. Pat. No. 6,139,851 and Cannell et al. in U.S. Pat. No. 6,013,250, both of which are incorporated herein by reference. For example, these hair care compositions can be aqueous, alcoholic or aqueous-alcoholic solutions, the alcohol preferably being a monohydric alcohol such as ethanol or isopropanol, in a proportion of from about 1 to about 75% by weight relative to the total weight for the aqueous-alcoholic solutions. The hair care compositions may also contain polyhydric alcohols including, but not limited to, ethylene glycol, propylene glycol, 1,3 butylene glycol, glycerine, sorbitol, and polyethylene glycol. Additionally, the hare care compositions may contain one or more conventional cosmetic or dermatological additives or adjuvants, as described supra.

In another embodiment of the present invention, the compositions are hair coloring compositions. Hair coloring compositions are herein defined as compositions for the coloring, dyeing, or bleaching of hair. The hair coloring compositions of the present invention comprise an effective amount of modified soy protein in a cosmetically acceptable medium. An effective amount of a water-soluble modified soy protein for use in a hair coloring composition is herein defined as a proportion of from about 0.001 to about 60% by weight relative to the total weight of the composition. These hair coloring compositions contain hair coloring active substances, which include, but are not limited to, oxidative dyes, direct dyes, or a mixture thereof, and peroxygen-based oxidizing agents for the bleaching of hair.

Oxidative dyes, which provide hair color that lasts for about 4 to 6 weeks, contain a mixture of a peroxygen-based oxidizing agent and an oxidizable coloring agent. Suitable peroxygen-based oxidizing agents include, but are not limited to, inorganic peroxygen oxidizing agents such as hydrogen peroxide, preformed organic peroxyacid oxidizing agents, organic peroxide oxidizing agents, and mixtures thereof. The oxidative hair coloring agents are formed by oxidative coupling of primary intermediates, including but not limited to p-phenylenediamines, N-alkylated p-phenylenediamines, alkyoated p-phenylenediamines, p-aminophenols, p-diaminopyridines, hydroxyindoles, aminoindoles, aminothymidines, or cyanophenols, with secondary intermediates, including, but not limited to phenols, resorcinols, m-aminophenols, m-phenylenediamines, naphthols, pyrazolones, hydroxyindoles, N-alkylated hydroxyindoles, esterified hydroxyindoles, catechols or pyrazoles. The concentration of each oxidizing hair coloring agent is preferably at about 0.001 to about 3%, more preferably at about 0.01 to about 2% by weight of the total composition. The total combined level of oxidative hair coloring agents in the hair coloring compositions of this invention is preferably from about 0.001 to about 5%, more preferably from about 0.01 to about 4% by weight of the total composition.

Direct hair coloring active substances are preformed dyes, which do not require an oxidative effect in order to develop their color and are washed out of the hair more readily than the oxidative dyes. Suitable direct dyes include, but are not limited to, 4-hydroxypropylamino-3-nitrophenol, 4-amino-3-nitrophenol, 2-amino-6-chloro-4-nitrophenol, HC Blue 2, HC Yellow 4, HC Red 3, Disperse Violet 4, Disperse Black 9, HC Blue 7, HC Blue 12, HC Yellow 2, HC Yellow 12, Disperse Blue 3, Disperse violet 1 and mixtures thereof.

Other components of a cosmetically acceptable medium for hair coloring compositions are described by Dias et al., in U.S. Pat. No. 6,398,821 and by Deutz et al., in U.S. Pat. No. 6,129,770, both of which are incorporated herein by reference. For example, hair coloring compositions may contain oxidizing acids, alkali, sequestrants, stabilizers, thickeners, buffers, carriers, surfactants, solvents, antioxidants, polymers, and conditioners, as described supra.

The hair coloring compositions of the present invention may be prepared from an aqueous alkaline solution comprising an effective amount of a modified soy protein, as described supra, in a cosmetically acceptable medium for hair coloring compositions, and a basic substance to raise the pH to between about 8.0 to about 12.0. These basic substances include, but are not limited to, ammonium hydroxide, sodium hydroxide, and potassium hydroxide. This aqueous alkaline composition may be used to prepare oxidative hair coloring, direct hair coloring, and bleaching compositions by combining it with a suitable oxidative or direct dye or a peroxygen-based oxidizing agent, as described supra.

The present invention also comprises a method for forming a protective film of modified soy protein on skin or hair by applying one of the compositions described above comprising an effective amount of modified soy protein to the skin or hair and allowing the formation of the protective film. The compositions of the present invention may be applied to the skin or hair by various means, including, but not limited to, spraying, brushing, and applying by hand. The modified soy protein-containing composition is left in contact with the skin or hair for a period of time sufficient to form the protective film, preferably for at least about 0.1 to 60 min. The present invention also comprises a method for coloring hair comprising applying a hair coloring composition comprising an effective amount of a modified soy protein and at least one hair coloring active substance to the hair by means described supra. This hair coloring composition is allowed to contact the hair for a period of time sufficient to cause coloration of the hair, preferably between about 5 to about 50 min. The hair coloring composition is then rinsed from the hair.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

All ingredients used in the preparation of the personal care compositions described in the following Examples are available commercially unless otherwise noted.

The meaning of abbreviations used is as follows “wt %” means percent by weight, “qs” means as much as suffices, “EDTA” means ethylenediamine tetraacetate, “CFTA” means the Cosmetic, Toiletry and Fragrance Association, “POE” means polyoxyethylene, “PEG” means polyethylene glycol, “cSt” means centistokes.

Applicants specifically incorporate the entire content of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

### EXAMPLE 1

### Hair Coloring Composition

A composition for oxidative dyeing of hair is made by mixing the following ingredients:

| | wt % |
|---|---|
| Ammonium lauryl sulfate (anionic surfactant) | 2.00 |
| Propylene glycol (moisturizer) | 4.00 |
| Ethoxydiglycol (solvent) | 2.00 |
| Monoethanolamine (pH adjuster) | 5.00 |
| Seaweed extract (conditioner) | 0.80 |
| Tetrasodium EDTA (chelating agent) | 0.80 |
| Isoascorbic acid (antioxidant) | 0.20 |
| Sodium sulfite (reducing agent) | 0.50 |
| Primary intermediates and couplers (dye) | 5.00 |
| Oleic acid (soap) | 12.50 |
| Cetearyl alcohol (opacifier) | 4.00 |
| Emulsifying wax (emulsifier) | 2.00 |
| Oleth-20 (nonionic surfactant) | 1.00 |
| Steareth-21 (nonionic surfactant) | 0.70 |
| Meadowfoam seed oil (oil) | 0.75 |
| Oleyl alcohol (oil) | 0.40 |
| Polyquaternium-10 (cationic surfactant) | 0.20 |
| Polyquaternium-28 (cationic surfactant) | 0.50 |
| Mica/titanium dioxide (pigment) | 0.30 |
| Water-soluble silk protein¹ | 1.00 |
| Cibafast ® W liquid² (UV absorber) | 1.00 |
| Fragrance | 0.75 |
| Ammonium hydroxide (pH adjuster) | 5.00 |
| Wheat amino acids solution | 1.00 |
| Hydrolyzed soy protein isolate (350 kDa), phthalic | 0.05 |
| anhydride-oxidized adduct³ | |
| Water | qs to 100 |

¹The water-soluble silk protein is prepared as described in copending U.S. Provisional Patent Application No. 60/425,617(in particular, Example 1), incorporated herein by reference.
²Cibafast ® W Liquid is sodium isobutyl benzotriazole sulfonate, obtained from Ciba Specialty Chemicals, Basil, Switzerland.
³This modified soy protein is prepared as described in U.S. Pat. No. 4,961,788 (Example 1).

The composition is made by first dissolving the first eight ingredients in water. The primary intermediates and couplers are then added with heat to dissolve. The remaining ingredients, except for the ammonium hydroxide, wheat amino acids, modified soy protein, and fragrance, are mixed separately and added after the primary intermediates and couplers. Then, the remaining water, ammonium hydroxide, wheat amino acids, modified soy protein, and fragrance are finally added to the mixture.

### EXAMPLE 2

### Aqueous Alkaline Composition

An aqueous alkaline composition suitable for mixing with a peroxide composition to form a hair bleach is prepared by mixing the following ingredients:

| | wt % |
|---|---|
| Erythrobic acid | 0.20 |
| Sodium sulfite | 0.50 |
| Propylene glycol | 4.00 |
| Ethoxydiglycol | 2.00 |
| Tetrasodium EDTA (38% aqueous Solution) | 0.80 |
| Ethanolamine | 5.00 |
| Hypnea musciformis extract/gellidiela acerosa/ | 0.80 |
| extract, sargassum filipendula extract/sorbitol | |
| Sodium benzotriazole sulfonate/buteth-3/propane | 1.00 |
| tricarboxylic acid | |
| Ammonium lauryl sulfate (28% aqueous solution) | 2.00 |
| Oleic acid | 12.50 |
| Cetearyl alcohol | 4.00 |
| Emulsifying wax | 2.00 |
| Oleth-20 | 1.00 |
| Steareth-21 | 0.70 |
| Meadowfoam seed oil | 0.75 |
| Oleyl alcohol | 0.40 |
| Polyquaternium-10 | 0.20 |
| Polyquaternium-28 | 0.50 |
| Mica/titanium dioxide (67:33) | 0.30 |
| Water-soluble silk protein¹ | 1.00 |
| Hydrolyzed soy protein isolate (150 kDa), phthalic | 1.00 |
| anhydride-oxidized adduct² | |
| Fragrance | 0.75 |
| Ammonium hydroxide (27.5% aqueous solution) | 5.00 |
| Water | qs to 100 |

¹The water-soluble silk protein is prepared as described in copending U.S. Provisional Patent Application No. 60/425,617 (Example 1).
²This modified soy protein is prepared as described in U.S. Pat. No. 4,961,788 (Example 1).

This composition is useful for creating several hair coloring formulations, including oxidative hair coloring, direct deposit hair coloring, bleaching cremes and permanent waving compositions.

### EXAMPLE 3

### Hair Bleach Composition

An emulsion is formed by mixing equal volumes of the aqueous alkaline composition of Example 2 and an aqueous hydrogen peroxide solution (6 wt %). This mixture is applied to natural light brown hair. The composition is left on the hair for five minutes, and then rinsed out with water. The resulting hair is lightened several shades.

### EXAMPLE 4

### Hair Shampoo

A hair shampoo is prepared by mixing the following ingredients:

| CFTA Names | wt % |
|---|---|
| Self emulsifying glyceryl ester | 6.0 |
| Cetrimonium chloride | 3.5 |
| Dicetyldimonium chloride | 3.0 |
| Water-soluble silk protein¹ | 0.1 |
| Cetearyl alcohol | 2.0 |
| Trimetylsyllamodimethicone | 0.7 |
| Ammonium lauryl sulfate | 25 |
| Cocamidopropyl betaine | 10 |
| Sodium lauryl sarcosinate | 5.0 |
| Hydrolyzed soy protein isolate (350 kDa), phthalic | 0.05 |
| anhydride-oxidized adduct² | |
| Water | qs to 100 |

¹The water-soluble silk protein is prepared as described in copending U.S. Provisional Patent Application No. 60/425,617 (Example 1).
²This modified soy protein is prepared as described in U.S. Pat. No. 4,961,788 (Example 1).

To 60 g of deionized water, each ingredient is added serially with moderate agitation until completely dissolved.

### EXAMPLE 5

### Hair Conditioner

A hair conditioner is prepared by mixing the following ingredients:

| CFTA Names | wt % |
|---|---|
| Self emulsifying glyceryl ester | 6.0 |
| Cetrimonium chloride | 3.5 |
| Dicetyldimonium chloride | 3.0 |
| Cetearyl alcohol | 2.0 |
| Hydrolyzed soy protein isolate (150 kDa), phthalic | 0.1 |
| anhydride-oxidized adduct¹ | |
| Trimethylsyllamodimethicone | 0.7 |
| Menthol | 0.1 |
| Phytolipid and hyaluronic Acid | 0.1 |
| Apricot seed (Apricot Kernel Powder produced by | 0.25 |
| Alban Muellen, Inc. of Paris, France) | |
| Pearlizing agent | 0.8 |
| Methyl gluceth-20 | 0.25 |
| Polyquaternium-4 | 0.1 |
| Water | qs to 100 |

¹This modified soy protein is prepared as described in U.S. Pat. No. 4,961,788 (Example 1).

To 55 g of deionized water heated to 60° C., the first 4 ingredients are added serially with moderate agitation until completely dissolved. The bulk solution is then cooled to 35° C., and the remaining ingredients are added serially with moderate agitation.

### EXAMPLE 6

### Hair Conditioner

A hair conditioner is prepared by mixing the following ingredients:

| CFTA Names | wt % |
|---|---|
| Self emulsifying glyceryl ester | 6.0 |
| Cetrimonium chloride | 3.5 |
| Dicetyldimonium chloride | 3.0 |
| Cetearyl alcohol | 2.0 |
| Hydrolyzed soy protein isolate (150 kDa), | 0.05 |
| phthalic anhydride-oxidized adduct¹ | |
| Hydrolyzed soy protein (unmodified)² | 0.05 |
| Trimethylsyllamodimethicone | 0.7 |
| Menthol | 0.1 |
| Phytolipid and hyaluronic Acid | 0.1 |
| Apricot seed (Apricot Kernel Powder produced by | 0.25 |
| Alban Muellen, Inc. of Paris, France) | |
| Pearlizing agent | 0.8 |
| Methyl gluceth-20 | 0.25 |
| Polyquaternium-4 | 0.1 |
| Water | qs to 100 |

¹This modified soy protein is prepared as described in U.S. Pat. No. 4,961,788 (Example 1).
²Hydrolyzed, unmodified soy protein is commercially available from many sources, including Croda, Inc., Parsippany, NJ.

To 55 g of deionized water heated to 60° C., the first 4 ingredients are added serially with moderate agitation until completely dissolved. The bulk solution is then cooled to 35° C., and the remaining ingredients are added serially with moderate agitation.

### EXAMPLE 7

### Emulsions for Skin Care

Three variations of skin emulsion compositions are prepared by mixing the following ingredients:

| | Comp. #1 | Comp. #2 | Comp. #3 |
|---|---|---|---|
| | wt % | wt % | wt % |
| Mineral Oil (A) | 4 | 4 | 4 |
| Petroselinic acid | 1.15 | 2 | 3 |
| (triglyceride) ex Elysion (A) | | | |
| Green Tea Polyphenols (A) | 0 | 2 | 0 |
| Hydrolyzed soy protein isolate | 0.001 | 0.01 | 1 |
| (250 kDa), phthalic anhydride- | | | |
| oxidized adduct¹ (A) | | | |
| Quercetin (A) | 0.5 | 0 | 0 |
| Brij 56² (A) | 4 | 4 | 4 |
| Alfol 16RD³ (A) | 4 | 4 | 4 |
| Triethanolamine (B) | 0.75 | 0.75 | 0.75 |
| Butane-1,3-diol (B) | 3 | 3 | 3 |
| Xanthan gum (B) | 0.3 | 0.3 | 0.3 |
| Perfume (C) | qs | qs | qs |
| Butylated hydroxy-toluene (B) | 0.01 | 0.01 | 0.01 |
| Water (B) | qs to 100 | qs to 100 | qs to 100 |

¹This modified soy protein is prepared as described in U.S. Pat. No. 4,961,788 (Example 1).
²Brij 56 is cetyl alcohol POE (10).
³Alfol 16RD is cetyl alcohol.

In separate kettles fitted with means for heating, materials for phase A and phase B are blended as follows. Materials for phase A, as indicated in parentheses above, are blended at 70° C. with moderate agitation until completely blended. Materials for phase B, as indicated in parenthesis above, are blended by heating 70 g of deionized water to 70° C. and adding the remaining materials serially. When both phases are clear they are blended together at high shear until a white composition is obtained. The composition is allowed to cool to 35° C. and the perfume (phase C) is added with moderate stirring.

### EXAMPLE 8

### Thicker Emulsions for Skin Care

Three variations of thicker emulsion compositions are prepared by mixing the following ingredients:

| Full Chemical or | Comp. #1. | Comp. #2. | Comp. #3. |
|---|---|---|---|
| Trade Name | wt % | wt % | wt % |
| Coriander seed oil ex (B) | 2 | 3 | 1.5 |
| Loders Croklaan (PA | 1.5 | 2.0 | 3.0 |
| triglyceride 60-75% of total | | | |
| fatty acids) (A) | | | |
| Hydrolyzed soy protein | 1 | 2 | 1.5 |
| isolate (250 kDa), phthalic | | | |
| anhydride- oxidized adduct¹ | | | |
| (B) | | | |
| Disodium EDTA | 0.05 | 0.05 | 0.05 |
| (Sequesterene Na₂) (B) | | | |
| Veegum ® Ultra Magnesium | 0.6 | 0.6 | 0.6 |
| Aluminum silicate (B) | | | |
| Methyl paraben (B) | 0.15 | 0.15 | 0.15 |
| Simethicone (B) | 0.01 | 0.01 | 0.01 |
| Butylene glycol 1,3 (B) | 3.0 | 3.0 | 3.0 |
| Hydroxyethylcellulose (B) | 0.5 | 0.5 | 0.5 |
| Glycerine, USP (B) | 2.0 | 2.0 | 2.0 |
| Xanthan gum (B) | 0.2 | 0.2 | 0.2 |
| Triethanolamine (B) | 1.2 | 1.2 | 1.2 |
| Stearic acid (A) | 3.0 | 3.0 | 3.0 |
| Propyl paraben NF (B) | 0.1 | 0.1 | 0.1 |
| Glyceryl hydrostearate (A) | 1.5 | 1.5 | 1.5 |
| Stearyl alcohol (A) | 1.5 | 1.5 | 1.5 |
| Isostearyl palmitate (A) | 6.0 | 6.0 | 6.0 |
| C12-15 alcohols (A) | 3.0 | 3.0 | 3.0 |
| Octanoate (A) | 3.0 | 3.0 | 3.0 |
| Dimethicone (50 cSt) (A) | 1.0 | 1.0 | 1.0 |
| Cholesterol NF (A) | 0.5 | 0.5 | 0.5 |
| Sorbitan stearate (A) | 1.0 | 1.0 | 1.0 |
| Butylated- | 0.05 | 0.05 | 0.05 |
| hydroxytoluene (B) | | | |
| Tocopheryl acetate (B) | 0.1 | 0.1 | 0.1 |
| PEG-100 stearate (A) | 2.0 | 2.0 | 2.0 |
| Sodium stearoyl lactylate (B) | 0.5 | 0.5 | 0.5 |
| Hydroxycaprylic acid (B) | 0.1 | 0.1 | 0.1 |
| Alpha-bisabolol (B) | 0.2 | 0.2 | 0.2 |
| Water(B) | qs to 100 | qs to 100 | qs to 100 |

¹This modified soy protein is prepared as described in U.S. Pat. No. 4,961,788 (Example 1).

In separate kettles fitted with means for heating, materials for phase A and phase B were blended as follows. Materials for phase A, as indicated in parentheses above, are blended at 70° C. with moderate agitation until completely blended. Materials for phase B, as indicated in parentheses above, are blended by heating 70 g of deionized water to 70° C. and adding the remaining materials serially. When both phases are clear they are blended together at high shear until a white composition is obtained. The composition is allowed to cool to 35° C.

### EXAMPE 9

### Facial Creme

A facial creme was prepared by mixing the following ingredients:

| Phase | Ingredient | Wt % |
|---|---|---|
| A | Water | 78.8 |
| A | Glycerin | 7 |
| A | Hydrolyzed soy protein isolate (250 kDa), phthalic | 1 |
| | anhydride-oxidized adduct¹ | |
| B | Isohexadecane | 3 |
| B | Polyacrylaminde (and) C13-14 Isoparaffin (and) | 2.5 |
| | laureth-7 | |
| B | Dimethicone & Dimethiconol | 2 |
| B | Isopropyl Isostearate | 1.3 |
| B | Sorbitan Stearate and Sucrose Cocoate | 1 |
| B | Cetyl Alcohol | 0.8 |
| B | Sucrose Polycottonseedate | 0.7 |
| C | Titanium Dioxide (and) water (and) glycerin (and) | 0.6 |
| | ammonium polyacrylate (and) parabens | |
| C | Benzyl Alcohol | 0.3 |
| C | PEG-100 Stearate | 0.2 |
| C | Methyl Paraben | 0.1 |
| C | Ethyl Paraben | 0.1 |
| C | Stearyl Alcohol | 0.6 |
| TOTAL | | 100 |

¹This modified soy protein was prepared as described in U.S. Pat. No. 4,961,788 (Example 1).

Phase A was blended in a kettle fitted with means for heating. Phase B was blended at 70° C. in a separate kettle fitted with means for heating until homogeneous. Phase C was added to Phase B. Then, Phases A and B were blended under high shear until uniform, and the mixture was cooled to room temperature with stirring.

1. A personal care composition comprising:
an effective amount of a soy protein selected from the group consisting of soy proteins modified by treatment with a reducing agent and subsequently reacted with a carboxylic acid anhydride; soy proteins modified by treatment with a reducing agent, reacted a carboxylic acid anhydride and subsequently oxidized; soy proteins modified by reaction with a hydroxy alkyl acrylate; soy proteins modified with epoxide, acrylate, or chlorohydrin ionic monomers; soy proteins modified by reaction with an alkyl acrylimidoglycolate; and mixtures thereof.

2. The composition of claim 1 further comprising at least one cosmetic adjuvant selected from the group consisting of fillers, surfactants, thixotropic agents, antioxidants, preserving agents, dyes, pigments, fragrances, thickeners, vitamins, hormones, moisturizers, UV absorbing organic sunscreens, UV scattering inorganic sunscreens, wetting agents, cationic, anionic, nonionic or amphoteric polymers, and hair coloring active substances.

3. The composition of claim 1 wherein the effective amount of the soy protein is from about 0.001 to about 90% by weight of the total weight of the composition.

4. The composition of claim 1 wherein the effective amount of the protein is from about 0.001 to about 60% by weight of the total weight of the composition.

5. The composition of claim 1 wherein the effective amount of the soy protein is from about 0.005 to about 20% by weight of the total weight of the composition.

6. The composition of claim 1 wherein the composition further comprises an effective amount of a natural or recombinant protein, or a hydrolyzed natural or recombinant protein.

7. The composition of claim 6 wherein the effective amount of the natural or recombinant protein is from about 0.001 to about 60% by weight of the total weight of the composition.

8. The composition of claim 6 wherein the effective amount of the natural or recombinant protein is from about 0.01 to about 20% by weight of the total weight of the composition.

9. The composition of claim 6 wherein the natural or recombinant protein is selected from the group consisting of wheat, oat, and rice proteins, collagen, keratins, gelatin, elastin, fibronectin, soluble reticulin, unmodified soy proteins in unhydrolyzed or hydrolyzed form, and water-soluble silk proteins.

10. The composition of claim 1, which is an anhydrous composition and contains 10 to 90% by weight, relative to the total weight of the composition, of a fatty phase, wherein the fatty phase contains at least one liquid, solid or semi-solid fatty substance.

11. The composition of claim 10, wherein the fatty substance is selected from the group consisting of isododecane, hydrogenated polyisobutene, squalane, isononyl isononanoate, cyclotetra- and -pentadimethicones, phenyltrimethicone, ethylene homopolymers, ethoxylated fats and oils, fluoroalkanes, microcrystalline waxes, ozocerite, beeswax, seracite, shea butter, candelilla wax, arachidyl propionate, fluoropolymers represented by the monomer:
X₁X₂C═CX₃F
wherein X₁, X₂, and X₃ are independently H or F,
and copolymers of ethylene and of at least one monomer represented by the formula:
CH₂═CH—R₃
wherein R₃ is an alkyl radical containing from 1 to 30 carbon atoms or an aryl or aralkyl radical.

12. The composition of claim 1, which is in the form of an aqueous solution containing mono or polyhydric alcohols.

13. The composition of claim 1, which is in the form of a creme emulsion, a gel, a dry powder, an aerosol, a mousse, an alcohol-in-oil emulsion, an alcohol and water solution, an aqueous solution, or an emulsion solution.

14. The composition of claim 13 further comprising a carbomer, gum, or other thickener in a proportion of from about 0.1 to about 15% by weight relative to the total weight of the composition.

15. The composition of claim 13 wherein at least one of the materials is in the form of a powder and wherein the effective amount of the soy protein is present along with a pigment or filler.

16. The composition of claim 15 wherein the effective amount of the soy protein is from about 0.001 to about 60% by weight of the total weight of the composition.

17. The composition of claim 15 wherein the pigment is selected from the group consisting of titanium dioxide, zinc oxide, barium oxide, D&C Red No.36 and D&C Orange No.17, the calcium lakes of D&C Red Nos. 7, 11, 31 and 34, the barium lake of D&C Red No.12, the strontium lake D&C Red No.13, the aluminum lakes of FD&C Yellow No. 5, of FD&C Yellow No.6, of D&C Red No.27, of D&C Red No.21, and of FD&C Blue No.1, iron oxides, manganese violet, chromium oxide, ultramarine blue, and carbon black particles.

18. The composition of claim 15 wherein the filler is selected from the group consisting of calcium carbonate, aluminum silicate, calcium silicate, magnesium silicate, mica, talc, barium sulfate, calcium sulfate, powdered polyamides, powdered polyesters, powdered fluorinated alkanes, and other inert powdered plastics.

19. The composition of claim 1, which is in the form of an aqueous alkaline solution suitable for preparing compositions for coloring or bleaching hair.

20. The composition of claim 19 further comprising a direct hair dye.

21. The composition of claim 19 further comprising an oxidative hair dye.

22. The composition of claim 19 further comprising a peroxygen-based oxidizing agent for bleaching hair.

23. The composition of claim 1, which is in the form of a stable dispersion of water-in-oil or oil-in-water type, and comprises:
a) a fatty phase in a proportion of from about 0.1 to about 50% by weight relative to the total weight of the composition, wherein the fatty phase contains the soy protein in a proportion of from about 0.001 to about 90% by weight relative to the total weight of the composition;
b) an aqueous phase in a proportion of from about 50 to about 98.9% by weight relative to the total weight of the composition; and
c) at least one emulsifier in a proportion of from about 1 to about 10% by weight relative to the total weight of the composition.

24. The composition of claim 23, wherein the fatty substance is selected from the group consisting of isododecane, hydrogenated polyisobutene, squalane, isononyl isononanoate, cyclotetra- and -pentadimethicones, phenyltrimethicone, ethylene homopolymers, ethoxylated fats and oils, fluoroalkanes, microcrystalline waxes, ozocerite, beeswax, seracite, shea butter, candelilla wax, arachidyl propionate, fluoropolymers represented by the monomer:
X₁X₂C═CX₃F
wherein X₁, X₂, and X₃ are independently H or F,
and copolymers of ethylene and of at least one monomer represented by the formula:
CH₂═CH—R₃
wherein R₃ is an alkyl radical containing from 1 to 30 carbon atoms or an aryl or aralkyl radical.

25. The personal care composition of claim 1 selected from the group consisting of skin care products, skin cleansing products, make-up, facial lotions, crème moisturizers, body washes, body lotions, foot and hand cremes, lipsticks, eyeshadows, foundations, facial powders, deodorants, shaving creme compositions, nail polishes, shaving lotions, creme depilatories, lotion depilatories, facial masks, anti-aging products, hair care compositions, shampoos, hair conditioners, hair treatment creams, styling gels, styling foams, hair mousses, hair sprays, set lotions, blow-styling lotions, hair color lotions, hair relaxing compositions, permanent wave first agents and permanent wave second agents.

26. A method for forming a protective film of soy protein on skin, hair, lips, or nails comprising:
a) applying to the skin, hair, lips, or nails the composition of claim 1; and
b) allowing the formation of the soy protein protective film on the skin, hair, lips, or nails.

27. A method for coloring or bleaching hair comprising applying the composition of claim 19, which further comprises dye or bleaching agent, to the hair for a period of time sufficient to cause the coloration of the hair.

28. The method of claim 27 wherein the composition is applied to the hair for a period of about 5 to about 50 minutes.
